(19) United States
(12) Patent Application Publication
(10) Pub. No.: US 2020/0222226 A1
(43) Pub. Date: Jul. 16, 2020

(54) Porlable Urinal For the Frequent Urrinator or The Disabled

(71) Applicant: Joshua Levine, Alexandria, VA (US)

(72) Inventor: Joshua Levine, Alexandria, VA (US)

(73) Assignee: Joshua Levine, Alexandria, VA (US)

(21) Appl. No.: 16/745,327

(22) Filed: Jan. 16, 2020

Related U.S. Application Data
(60) Provisional application No. 62/793,117, filed on Jan. 16, 2019.

(51) Int. Cl. A61F 5/44; A61F 5/445; B25J 13/08; B25J 11/00; F16L 37/10

(52) U.S. Cl. CPC A61F 5/4405; A61F 5/445; B25J 5/005; B25J 11/009; F16L 37/101; B25J 13/086

(57) ABSTRACT

A portable urinal designed to collect urine from one location and deposit that urine into another location for disposal (e.g. drain). The invention enables a person to remain sedentary while urinating and waste disposal.

### BACKGROUND SUMMARY OF INVENTION

Many people experience frequent urination. This urge can be especially troubling at night where frequent trips to the bathroom are disruptive to sleep. This machine is designed to decrease the time and activity involved in night lime urination and increase sleep time and restfulness.

### SUMMARY OF THE INVENTION

The invention is a portable urinal designed to collect urine from one location and deposit that urine into another location for disposal (e.g. drain). The invention enables a person to remain sedentary and have a urine collector delivered to them. The urine collector is moved by a robotic vehicle. The robotic vehicle initially may be located within an arm's reach of the person or may be summoned within arms reach of the person. When within the prescribed distance, the person may interface with the robotic vehicle containing the urine collector.

### DETAILED DESCRIPTION

### The Male and Female Quick Disconnect.

The Invention comprises at least a pair of automatically connecting quick disconnect connectors. A quick disconnect is a coupling used to provide a fast, make-or-break connection of fluid transfer lines. Quick connect fittings replace threaded or flanged connections, which require tools for connection of a leak free fluid fine. The pair of quick disconnects are held are different two locations, one location being for a male component and one location being for a female component. The male component is inserted into the female component via a first actuator. The female quick disconnect has a sleeve that needs to be retracted to allow for male insertion. This sleeve is connected to a spring which holds the female part closed with no external force. A bracket or covering is attached to the female sleeve The bracket or covering grasps the sleeve so that any force put on the covering or bracket is transferred to the sleeve. The sleeve or covering is connected to a second actuator that is adapted to drive the sleeve forwards and backwards. The second actuator may be a lead screw mounted to a motor. The motor may be electric and has a coupling shaft. The coupling shaft of the motor is connected to the lead screw via a flexible coupling. This flexible coupling allows movement at least one degree of freedom and up to all six degrees of freedom. This is important has the flexible coupling allows the sleeve or covering to move and correct for misalignment when coupling the male and female parts of the quick disconnect. The motor is mounted to a base and is static. The lead screw actuates the sleeve or covering to move backwards or forwards to either eject or receive the male component. The sleeve must be retracted a certain distance to free the coupling. The motor is attached to a microprocessor. The microprocessor is set up to run the motor as well as communicate with proximity sensors. The proximity sensors may be electric or mechanical. The motor continues to move the actuator until one of the proximity sensors is tripped. The tripped sensor sends a signal to the microprocessor. The microprocessor then shots off the motor. The sensors may be hall effect sensors or limit switches. The sheath is now sufficiently pulled back to either eject or accept the male. The male portion is then advanced into the female portion using the first actuator until a limit switch or sensor is tripped. Another method that may be used to retract the sheath is to use a stepper motor attached to the microprocessor. The stepper motor may be advanced or retreated a certain number of steps corresponding to the necessary distance the to retract or advance the sheath. Alternatively, the motor may include a feedback position sensor (e.g. position encoder) that can measure the distance of the sleeve is retracted as the motor turns. Additionally, some quick disconnects are simply inserted to connect without sleeve retraction. In this scenario the male quick disconnect would enter the female quick disconnect via an actuator until a proximity sensor is tripped. Upon detachment the sleeve on the female quick disconnect would be retracted via an actuator until a proximity sensor is tapped and the male quick disconnect is then able to be freely withdrawn.

### The Base Unit

The base unit resides close to a drain, e.g. toilet) and acts like a docking station for the robot. The base unit comprises a quick disconnect portion, docking sensor, and fluid outlet tube. When the robot docks at the base station, a docking sensor is activated and sends a signal to robot to engage the docking process and connect the quick disconnects. The docking sensor may be a mechanical device, magnetic device, optical device or electrical device. The signal may be a hardwired electrical signal or wireless signal. The base unit is connected to the drain. The quick disconnect portion is attached to a tube which in turn is run into a drain. The base station may be located within a bathroom. The base unit may comprise a pump to pump fluid out of a bag. The base station may be self cleaning and be attached to a fresh water supply. For example, a toilet's water supply may be replaced with a t-pipe, with one end of the t-pipe attached to the toilet hose and the other end of the t-supply attached to a hose of the base unit. The may unit may also comprise a tube that runs into a toilet bowl. More specifically, the tube may run under the spacers of a toilet seat.

### The Robot

The robot is s vehicle with multiple sensors designed to detect and navigate around obstacles. These sensors include accelerometers, mechanical collision detectors, optical sensors. The robot is designed with communication sensors to communicate with other communication sensors at different locations that may contribute to the guidance of its movements. The robot may include wheels, tracks or other means for locomotion. The robot is configured to carry and move a bag of fluid from one location to other locations. A quick disconnect assembly is mounted inside or on top of the robot. The robot has motors attached to its wheel that drive the locomotion. The robot may have a pump to pump the fluid from the bag externally. The robot is also equipped with a vertical actuator designed to lift an interface portion of the bag upwards so it may deliver it within an arm's reach of a patient.

### The Portable Urinal

The portable urinal is comprised of a bag to contain the urine, a portion configured to conform the anatomy of the patient (to fit over the penis or seal over the vagina) and a tube connecting the urine bag to one of the quick disconnects. A backflow valve sits between the urine bag and the anatomically conforming portion and prevents urine from backflowing into the anatomical portion to hold it safely inside the urine bag. The connection between the tube and quick disconnect portion is a fluid connection that as a waterproof, fluid seal. The portable urine resides on the robot and is shuttled from the patient's position to the base units position.

### The Drain

The drain is connected to pipes that connect to a sewage system. The base unit has a tube that is either directly or indirectly connected to the drain.

What is claimed is:

1. A portable urine collection device comprising:
a urethra interface connection configured to form a seal with a urethra, a first hollow tube with at least two ends, with one end of the first hollow tube connected to the urethra interface connection, a hollow bladder that connects to the first hollow tube, a second hollow tube connected to the hollow bladder and wherein another end of the second hollow tube is connected to a valve.
